(19) United States
(12) Patent Application Publication
(10) Pub. No.: US 2025/0177154 A1
(43) Pub. Date: Jun. 5, 2025

(54) Method for simulating the joint of a patient

(71) Applicant: FX SHOULDER SOLUTIONS, Viriat (FR)

(72) Inventors: Jean-Marie DAUDET, Viriat (FR); Baptiste MARTIN, Viriat (FR)

(73) Assignee: FX SHOULDER SOLUTIONS, Viriat (FR)

(21) Appl. No.: 18/963,600

(22) Filed: Nov. 28, 2024

(30) Foreign Application Priority Data
Nov. 30, 2023 (FR) FR2313379

(51) Int. Cl. A61F 2/40; A61B 34/10

(52) U.S. Cl. CPC A61F 2/4059; A61B 2034/102; A61B 2034/105

(57) ABSTRACT

Method for simulating a joint of a patient in order to plan joint prosthesis 20 implantation surgery including: analyzing an image 1 of the patient's joint to extract geometric information concerning the geometry of the bones 3, and of some at least of the soft tissues 2 forming it; fusing this information, assigning geometrical properties to the bones; using this information, assigning to the soft tissues of the points of connection on the bones, as well as connection forces estimated using the dimensions of these tissues; digitally modeling the joint taking into account the geometrical properties of the bones and the points of connection of the soft tissues, as well as the maximum connection forces of the soft tissues; integrating a digital simulation of the joint prosthesis 20 to the digital model; animating the digital model obtained after integrating the prosthesis, to anticipate its effect of the patient receiving surgery.

### FIELD OF THE INVENTION

The invention relates to the field of joint prosthesis implants.

More specifically, the invention relates to a method for simulating the joint of a patient in order to plan joint prosthesis implant surgery, for example a shoulder prosthesis.

### BACKGROUND OF THE INVENTION

It is already known, in the state of the art, to produce a customized model of part of a skeleton of a patient who is to receive a joint prosthesis after taking a scan of the targeted joint. The model is used to determine the geometry of the joint and choose the prosthesis model that is the most likely to restore the movement capacities of the patient after surgery. The authorized movements depend on the geometry of the skeleton and the mechanical connections implemented by the prosthesis.

One difficulty of the method of the state of the art is that, despite the precautions taken by precise pre-operative planning, we observe that the patients do not all benefit in the same way from the benefits of the prostheses even though the models are identical. There are therefore other factors which influence the result of the implantation.

As a first assumption, it has been proposed that diseases specific to each patient may create differences which influence the results of the prosthesis implantation.

The inventor has made and checked an additional assumption to explain this disparity of results between the patients. The soft tissues such as ligaments, tendons, muscles and cartilage, which are not taken into account in the simulation, have a significant influence on the success of the implantation. If they were taken into account, the simulation would lead to potentially different choices of the prosthesis model and of the implantation of the prosthesis on the skeleton, in order to obtain a more uniform result from one patient to another. Following the tests conducted by the inventor, this result has proved to be true.

In this description, “peri-bone tissue” designates all the soft tissues and the cartilage that are in contact with the bones of a joint.

### SUMMARY OF THE INVENTION

Thus, the invention relates to a method for simulating a joint of a patient in order to plan joint prosthesis implantation surgery, the method consisting in:
obtaining an image of the joint containing point clouds characteristic of the geometry not only of the patient's bones forming the joint, but also of at least some of the peri-bone tissues forming said joint, said peri-bone tissues consisting of soft tissues and/or cartilage that are in contact with the patient's bones forming the joint,
submitting the image containing the point clouds to shape recognition, by comparison with a data bank of anatomical parts including information concerning the points of insertion on the bones and the muscle connection forces, and/or, using deep learning and artificial intelligence, to:
identify the bones and the peri-bone tissues forming the joint, and assign them properties concerning the points of insertion on the bones and the connection forces,
creating a “segmented” digital model of the joint taking into account the bones and the peri-bone tissues identified and their properties so obtained.
integrating a digital simulation of joint prosthesis to the segmented digital model obtained during the preceding step, replacing the anatomical parts to be replaced by the prosthesis,
animating the digital model obtained after integration, to anticipate the effect of the presence of said prosthesis on the patient receiving surgery.

Advantageously, the peri-bone tissues comprise one at least from the set composed of the muscles, the tendons, the ligaments, the cartilage.

In a first embodiment of the invention, the image is obtained by a scanner, then processed to reveal the soft tissues which are not normally easily visible on a raw scanner image.

According to another embodiment, the image is obtained by MRI (magnetic resonance imaging).

According to the invention, according to a first model, the digital model made using the image consists in identifying remarkable points of the image to form point clouds and to build segments representative of the components of the model. This step, which consists in converting a point cloud into a solid, is called segmentation.

According to the invention, according to a second model, a finite elements representation is used. Each bone and each peri-bone tissue is represented by a mesh of elementary structures.

According to a particular embodiment of the invention, in the segmented digital model, each muscle is broken down into a predetermined number of separate muscular fibers, each muscular fiber receiving a force value, which is a component of the overall force of the muscle. This force value may vary in the joint movement cycle.

According to a particular embodiment of the invention, the joint is a shoulder and the bones are the humerus, the scapula and the collarbone.

According to another particular embodiment, the joint is a hip or a knee.

According to a particular embodiment of the invention, the peri-bone tissues taken into account in the segmented digital model are the shoulder muscles, i.e. the rotator cuff, in other words supraspinatus and infraspinatus, subscapularis and teres minor, and the deltoid.

According to the invention, the insertion of ligaments on the bones is taken into account in the segmented digital model precisely, in order to simulate very precisely the pairs of forces exerted by the muscles on the bones.

Advantageously, in the segmented digital model, each muscle is independent of the others.

Advantageously, in the segmented digital model, each fiber of a muscle is independent of the other fibers.

In a particular embodiment of the invention, a particular pathology of the patient is simulated in the segmented digital model by assigning a degraded maximum force value to each fiber of a muscle and/or to each muscle.

In a particular embodiment of the invention, animating the digital model after integrating the prosthesis consists in digitally contracting each muscle or muscle fiber, in other words in simulating the application of a force by each muscle or muscle fiber on the bones and in observing the theoretical movement of the bones in their degrees of freedom allowed by the simulated joint prosthesis.

The invention can therefore be used to plan, in a concrete and realistic manner, the future results of the implantation of the prosthesis on the patient.

In a particular embodiment, the method according to the invention can also be used to predict which muscles will require development that will be advantageous for the optimum functioning of the joint. Thus, the practitioner will be able to recommend to the patient muscular training movements designed to develop certain muscles which will make the functioning of the joint equipped with the prosthesis more efficient. Such progress was not possible with the purely bone models of the state of the art.

The invention allows the surgeon to plan a surgical operation fully, taking into account not only the patient's local skeleton, but also the soft tissues. In the state of the art, the peri-bone tissues are not known and the surgeon only discovers at the start of the surgical operation the environment of the patient's skeleton, consisting of the muscles, tendons, ligaments and cartilage. The surgeon must therefore adapt to the situation in real time, in other words without having been able to prepare for it, and possibly adjust the attachment of the joint.

According to the invention, the purpose of applying contractions on muscle models of the digital model is to obtain simple movements such as: abduction adduction, flexion extension, internal or external rotation, circumvolutions

According to a particular embodiment of the invention, animation of the digital model is renewed using different assumptions of the patient's muscular development, in order to anticipate the behavior of the prosthesis not only in the context of the state of the patient's muscular development before surgery, but also in changing contexts of the patient's muscular development which would be recommended by the practitioner after surgery.

In a particular embodiment, the information obtained from the data bank comprises the bone density and/or quality. This improves the resistance of the prosthesis over time.

The invention also relates to the digital model of the local skeleton and of the peri-bone tissues surrounding the skeleton, obtained by implementing the method defined above.

The invention also relates to a method for selecting a prosthesis from those available, which consists in:
successively testing each of the prostheses available by implanting a digital model of the prosthesis and animating the digital model of the local skeleton and the peri-bone tissues surrounding the skeleton integrating said prosthesis according to a predetermined field of muscular forces,
comparing the results obtained,
when all the prosthesis models have been tested, selecting the model which gives the best results.

In a particularly advanced embodiment, the method consists in animating the digital model with several fields of muscular forces, each field of muscular forces representing a potential state of the patient's muscular development, in order to select the joint prosthesis which gives the best results in a given field of muscular force. In this assumption, after surgery, the patient will be recommended to take muscular training in order to develop the field of muscular forces adapted to the prosthesis so selected.

The invention also relates to a computer program product allowing surgical planning, comprising a series of instructions to implement one at least of the methods described above.

### BRIEF DESCRIPTION OF THE FIGURES

The invention will be better understood on reading the following description, given solely by way of example and with reference to the accompanying drawings in which:

FIG. 1 is a flowchart illustrating a method according to the invention;

FIG. 2 is a perspective and cross-sectional scanned view of a patient's shoulder, in which a scapular implant is shown opposite the corresponding glenoid;

FIG. 3 is a view similar to that of FIG. 2, in which the soft tissues have been removed by computer and in which the scapular implant is shown implanted on the scapula;

FIG. 4 is a view showing a humeral implant, implanted in one end of a humerus; and,

FIG. 5 is a view similar to those of FIGS. 2 and 3 in which the scapular and humeral implants are shown in a relative functional position.

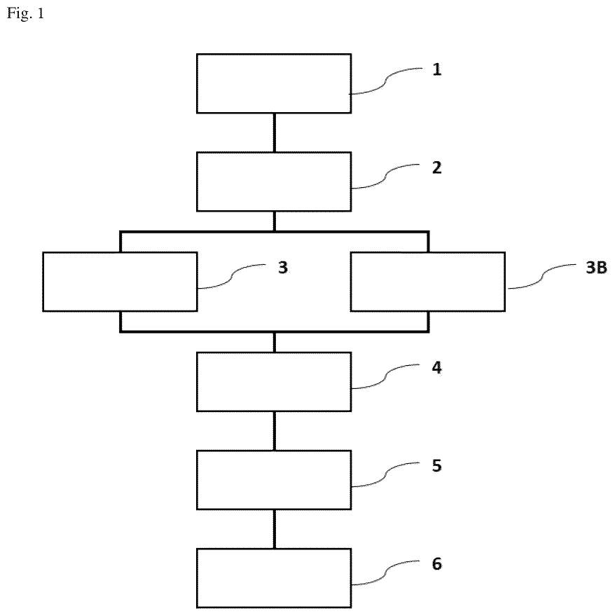

### DETAILED DESCRIPTION

During a first step 1, an image is taken of a patient's joint area, for example the shoulder. In this example, the imaging instrument used is a scanner.

During a second step 2, the images obtained undergo image processing which reveals in greater detail the geometry of the peri-bone tissues surrounding the patient's local skeleton, in order to reveal point clouds characteristic of these peri-bone tissues. This step is not necessarily required if the image obtained in step 1 is precise enough to immediately reveal the point clouds characteristic of the bones and the peri-bone tissues.

An image combining data from a scanner or an MRI instrument can also be used.

During a next step 3, the anatomical elements present on the image are recognized using artificial intelligence, which compares the point clouds of the image with representations of bones and of peri-bone tissues present in a data bank containing geometrical information concerning the anatomical parts and information concerning the points of insertion on the bones and on the muscle connection forces. A segmented digital model of the joint is thus obtained.

In a variant 3B of step 3, each muscle is replaced by ten independent muscular fibers, which undergo the same processing. In this case, the contraction of each muscle results in a contraction of each fiber, taking into account the individual properties of each fiber whose force, variable during the targeted movement, may have been adjusted manually to take into account a disease of the patient.

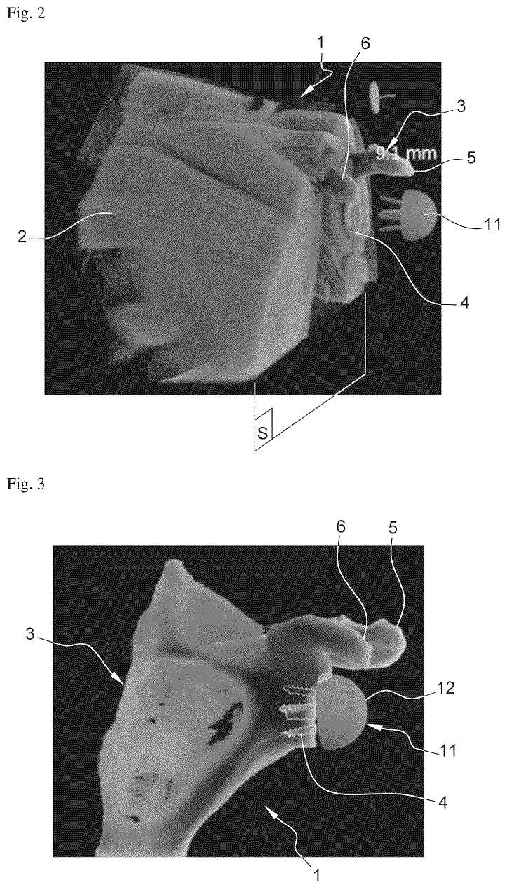

During a next step 4, a digital model of a joint prosthesis is selected and this digital model is integrated in the segmented digital model of the joint, digitally replacing the anatomical parts to be replaced by the joint prosthesis. This step 4 is illustrated by the images of FIGS. 2 to 5.

During a next step 5, the digital model is animated by applying forces on some muscles to simulate their contractions and the movement thus produced on the skeleton connected by the joint prosthesis is observed.

During a next step 6, the implantation of the joint prosthesis simulated in the preceding step is modified until optimum behavior of the joint is obtained. Optimum movement means a movement close to that of a healthy patient with similar morphology. Thus, the best location of the implant or implants and the best combination of implants to obtain the best results are determined. In addition, the necessary musculation to be developed by the patient after surgery, to optimize their recovery, is determined.

The finite elements model and the software resulting from implementing the method that has just been described can be produced using the general knowledge of those skilled in the art and will not be described here.

FIGS. 2 to 5 show the installation according to the invention of a reverse prosthesis. In the example shown, prosthesis of the scapulohumeral joint is installed.

FIG. 2 shows a 3D digital representation of the patient's left shoulder 1, in which the soft tissues, mainly muscular tissues 2, have been removed by computer on the right of a substantially vertical plane of section S. From these soft tissues 2, emerging from the plane of section S, parts of the scapula 3 can be seen to emerge, in particular: the glenoid 4, flush with the plane of section S, the acromion 5 and the coracoid 6. FIG. 2 also shows a digital model of scapular implant 11, shown away from the scapula 3. In the remainder of the document, the implant model will simply be called “implant”.

As shown in particular on FIG. 3, the scapular implant 11 is intended to be implanted on the scapula, at the position of the glenoid 4.

On the image 1 of FIG. 3, the soft tissues have been completely removed by computer from the representation of the shoulder, so that only the bone of the scapula and the scapular implant attached thereto can be seen.

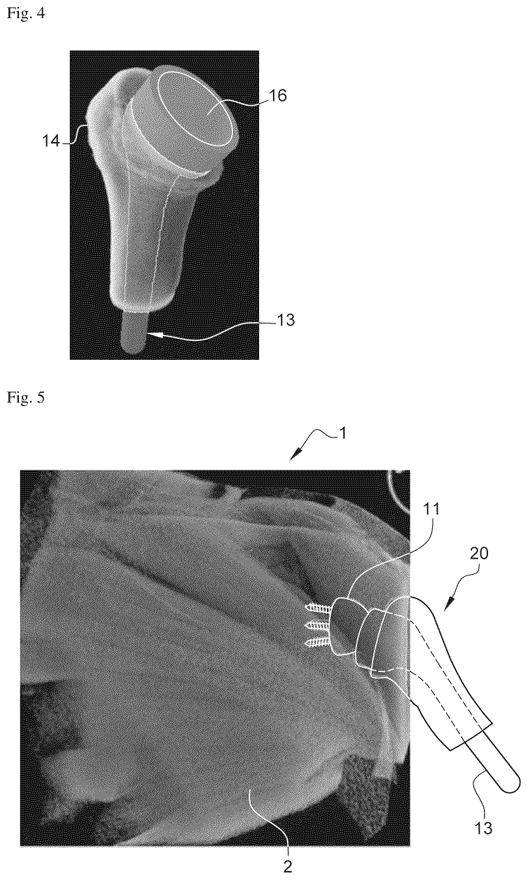

The scapular implant functionally replaces the glenoid, except that its joint surface 12 is convex. Thus, it is designed to form a reverse prosthesis 20, with a complementary humeral implant 13 of which a digital model can be seen on FIGS. 4 and 5.

FIG. 4 shows a 3D digital representation of the head 14 of the patient's left humerus 1, on which the soft tissues have been removed by computer. This figure also shows the implantation of the humeral implant 13, in the head 14 of the humerus. The implant 13 comprises a concave joint surface 16, complementary to the convex joint surface 12 of the glenoid. The sliding of the two surfaces 12, 16 over each other allows the joint 20 to function,

FIG. 5 is a digital representation of the joint 20, once reduced, in the configuration and in the bone and muscular context that it is likely to take during its actual implantation in the patient's shoulder. The 3D image of FIG. 5 therefore represents a digital twin of the patient's shoulder equipped with the prosthesis, before installing the prosthesis.

The digital twin can be deformed anatomically in order to model dynamic functioning of the prosthesis. It can be modified, for example by changing the shapes and dimensions of the implants, for example to choose those that are best adapted, fit the best in the anatomy and/or provide the greatest comfort of use for the patient.

This digital twin can therefore be used to plan the surgery. It can be used for example to modify, before the surgical operation, the shape of the scapular implant to prevent bone interference with some parts of the scapula.

This method, by installing a prosthesis that is more adapted, in terms of shape and position, to the patient's anatomy, results in better rehabilitation and better recovery. It will also make the joint more mobile.

Obviously, the invention is not limited to the examples which have just been described. On the contrary, the invention is defined by the following claims.

It will appear to those skilled in the art that various modifications can be made to the embodiments described above, in the light of the information that has just been disclosed.

Thus, the method described previously referring to a reverse prosthesis can be used for an anatomic prosthesis.

Thus, the method can be used for any type of joint prosthesis, for which it is important to check the functionality of the prosthesis in each of the positions it may take, and also, in its movement. It can also be used for non-mobile prostheses to check and limit interference with the patient's body.

1. A method for simulating a joint of a patient in order to plan joint prosthesis implantation surgery, the method comprising the steps of:
obtaining an image (1) of the joint containing point clouds characteristic of the geometry not only of the patient's bones (3, 14) forming the joint, but also of at least some of the peri-bone tissues forming said joint, said peri-bone tissues having soft tissues (2) and/or cartilage that are in contact with the patient's bones forming the joint,
submitting the image containing the point clouds to shape recognition, by comparison with a data bank of anatomical parts including information concerning the points of insertion on the bones and the muscle connection forces, and/or, using deep learning and artificial intelligence, to:
identify the bones and the peri-bone tissues forming the joint; and,
assign them properties concerning the points of insertion on the bones and the connection forces,
creating a “segmented” digital model of the joint taking into account the bones and the peri-bone tissues identified and their properties so obtained,
integrating a digital simulation (20; 11, 13) of joint prosthesis to the segmented digital model obtained during the preceding step, replacing the anatomical parts to be replaced by the prosthesis,
animating the digital model obtained after integration, to anticipate the effect of the presence of said prosthesis on the patient receiving surgery.

2. The method according to claim 1, wherein the peri-bone tissues including at least one selected from the set composed of the muscles, the tendons, the ligaments, the cartilage.

3. The method according to claim 1, wherein the image is obtained by a scanner and/or MRI, then processed to reveal the soft tissues.

4. The method according to claim 1, wherein, in the digital model, each muscle is broken down into a predetermined number of separate muscular fibers, each muscular fiber receiving a maximum force value, which is a component of the overall maximum force of the muscle, which may vary during an animation cycle of said model.

5. Th method according to claim 1, wherein the digital model made using the image including performing a segmentation, in other words in identifying remarkable points of the image to form point clouds and to build segments representative of the components of the model.

6. The method according to claim 5, wherein each muscle is broken down into a predetermined number of separate muscular fibers, each muscular fiber receiving a force value, which is a component of the overall force of the muscle, said force value possibly varying in the animation cycle of the model.

7. The method according to claim 1, wherein the digital model made using a representation by finite elements, each bone and each peri-bone tissue being represented by a mesh of elementary structures.

8. The method according to claim 1, wherein the joint is a shoulder and the bones are the humerus, the scapula and the collarbone.

9. The method according to claim 1, wherein the muscles are those of the shoulder, i.e. the rotator cuff, in other words supraspinatus and infraspinatus, subscapularis and teres minor, and the deltoid.

10. The method according to claim 1, wherein a particular pathology of the patient is simulated in the digital model by assigning a degraded maximum force value to each fiber of a muscle and/or to each muscle.

11. The method according to claim 1, wherein animating the digital model after integrating the prosthesis including digitally contracting each muscle or muscle fiber, in other words in simulating the application of a force by each muscle or muscle fiber on the bones and in observing the theoretical movement of the bones in their degrees of freedom allowed by the simulated joint prosthesis (20).

12. (canceled)

13. The method for selecting a prosthesis from those available, the method comprises the steps of:
successively testing each of the prostheses available by implanting a digital model of the prosthesis in a digital model and animating the digital model integrating said prosthesis according to a predetermined field of muscular forces,
comparing the results obtained,
when all the prosthesis models have been tested, selecting, preferably automatically, preferably using artificial intelligence, the model which gives the best results.

14. The method according to claim 13, wherein the digital model is animated with several fields of muscular forces, each field of muscular forces representing a potential state of the patient's muscular development, in order to select the joint prosthesis which gives the best results in a given field of muscular force.

15. A computer program product allowing surgical planning, comprising a series of instructions to implement one at least of the methods according to claim 13.
